(19) United States
(12) Patent Application Publication
(10) Pub. No.: US 2011/0130578 A1
(43) Pub. Date: Jun. 2, 2011

(54) STEREOSELECTIVE SYNTHESIS OF CERTAIN TRIFLUOROMETHYL-SUBSTITUTED ALCOHOLS

(75) Inventors: Daniel R. FANDRICK, Danbury, CT (US); Daniel KUZMICH, Danbury, CT (US); Jonathan T. REEVES, New Milford, CT (US); Jinhua J. SONG, Hopewell Junction, NY (US); Zhulin TAN, Cheshire, CT (US); Thomas LEE, Lexington, MA (US)

(73) Assignee: BOEHRINGER INGELHEIM INTERNATIONAL GMBH, Ingelheim am Rhein (DE)

(21) Appl. No.: 12/788,549

(22) Filed: May 27, 2010

Related U.S. Application Data
(60) Provisional application No. 61/183,601, filed on Jun. 3, 2009.

(51) Int. Cl. C07D 301/02

(52) U.S. Cl. 549/518

(57) ABSTRACT

A process for stereoselective synthesis of a compound of Formula (X) or Formula (X′)

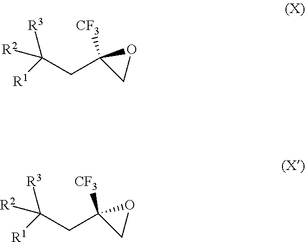

wherein:
R¹ is an aryl group substituted with one to three substituent groups,
   wherein each substituent group of R¹ is independently C₁-C₅ alkyl, C₂-C₅ alkenyl, C₂-C₅ alkynyl, C₁-C₅ alkoxy, C₁-C₅ alkoxycarbonyl, aminocarbonyl, alkylaminocarbonyl, dialkylaminocarbonyl, C₁-C₅ alkoxycarbonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, or C₁-C₅ alkylthio wherein the sulfur atom is oxidized to sulfoxide or sulfone, and
R² and R³ are each independently hydrogen or C₁-C₅ alkyl.

### FIELD OF THE INVENTION

The present invention relates to the stereoselective synthesis of certain trifluoromethyl-substituted alcohols.

### BACKGROUND OF THE INVENTION

Trifluoromethyl-substituted alcohols of formula (I) have been described as ligands that bind to the glucocorticoid receptor. These compounds are potential therapeutics in treating a number of diseases modulated by glucocorticoid receptor function, including inflammatory, autoimmune and allergic disorders. Examples of these compounds are described in U.S. Pat. Nos. 7,268,152; 7,189,758; 7,186,864; 7,074,806; 6,960,581; 6,903,215; and 6,858,627, which are each incorporated herein by reference in their entireties and are hereinafter termed “the Trifluoromethyl-Substituted Alcohol Patent Applications”.

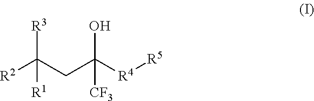

It is well known in the art that enantiomers of a particular compound can have different biological properties including efficacy, toxicity, and pharmacokinetic properties. Thus, it is often desirable to administer one enantiomer of a racemic therapeutic compound.

The synthetic methods disclosed in the patent applications cited above describe the synthesis of racemic products. Separation of enantiomers was accomplished by chiral HPLC and may be accomplished by other conventional ways of separating enantiomers. Chiral HPLC and other enantiomer separation method, however, are generally unsuitable for large-scale preparation of a single enantiomer. Thus, a stereoselective synthesis for preparation of these compounds would be highly desirable.

The present invention discloses a stereoselective synthesis of certain compounds of Formula (X) or (X′)

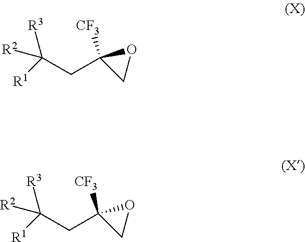

which are key intermediates in the synthesis of enantiomerically pure compounds of Formula (I).

The key step involves a diastereoselective addition of chiral sulfoxide anion to a trifluoromethyl ketone to form a chiral (β-hydroxy-β-trifluoromethyl-sulfoxide adduct. In the literature there are limited examples of such diastereoselective addition to fluorinated ketones, e.g., P. Bravo et al., J. Chem. Soc., Perkin Trans. I 1995, 1667; P. Bravo et al., J. Org. Chem. 1990, 55, 4216; C. Mioskowski and G. Solladie, Tetrahedron 1980, 36, 227. In these examples, the authors did not convert these adducts to the corresponding chiral epoxides. A. Arnone et al., Tetrahedron Lett. 1996, 37, 3903, describe an enzymatic reduction method for the synthesis of chiral β-hydroxy-β-trifluoromethyl thiol ether and its subsequent conversion to a chiral trifluoromethyl-substituted epoxide.

### SUMMARY OF THE INVENTION

The instant invention is directed to a process for stereoselective synthesis of a compound of Formula (X) or Formula (X′)

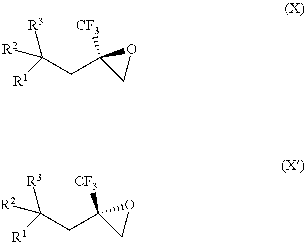

wherein:
R¹ is an aryl group substituted with one to three substituent groups,
wherein each substituent group of R¹ is independently C₁-C₅ alkyl, C₂-C₅ alkenyl, C₂-C₅ alkynyl, C₁-C₅ alkoxy, C₁-C₅ alkoxycarbonyl, aminocarbonyl, alkylaminocarbonyl, dialkylaminocarbonyl, C₁-C₅ alkoxycarbonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, or C₁-C₅ alkylthio wherein the sulfur atom is oxidized to sulfoxide or sulfone, and
   R² and R³ are each independently hydrogen or C₁-C₅ alkyl,
   the process comprising:
a) reacting the trifluoroacetamide of Formula (A) wherein R′ and R″ are each independently C₁-C₅ alkyl optionally substituted with O or N (e.g., morpholine amide or Weinreb amide) with a vinyl magnesium bromide bearing R² and R³ in a suitable solvent to provide the trifluoromethylenone of Formula (B)

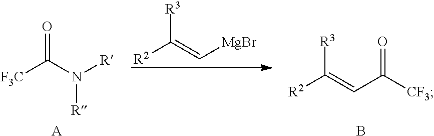

(b) reacting the trifluoromethylenone of Formula (B) with a suitable organocopper reagent generated from an organometallic reagent R⁵R⁴M where M is Li or MgX and a copper salt CuX, where X is Cl, Br, I, or CN, in a suitable solvent to form the ketone of Formula (C)

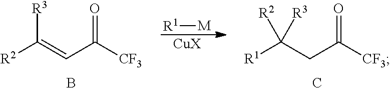

(c) reacting the ketone of Formula (C) with a chiral sulfoxide anion source (D) or (D′), where R is an alkyl or aryl group and M is a counter-cation, in a suitable solvent to prepare a compound of Formula (E) or (E′), respectively

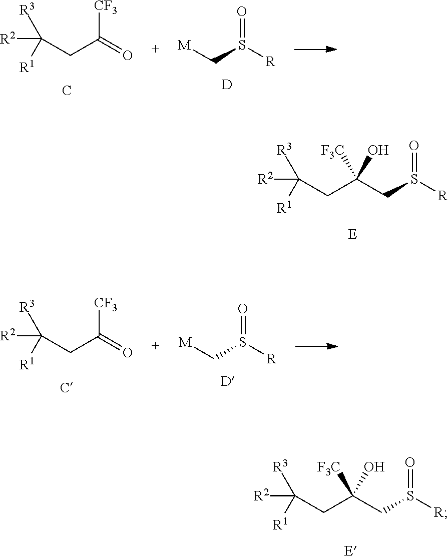

(d) reducing the sulfoxide of Formula (E) or (E′) in a suitable solvent to obtain the compound of Formula (F) or (F′), respectively

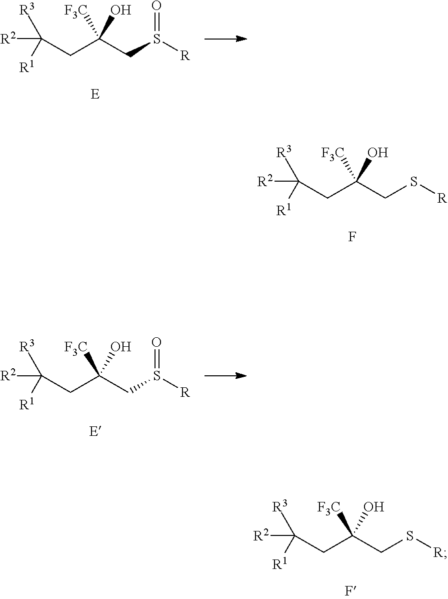

and
(e) cyclizing the compound of Formula (F) or (F′) in a suitable solvent to form the epoxide compound of Formula (X) or Formula (X′), respectively

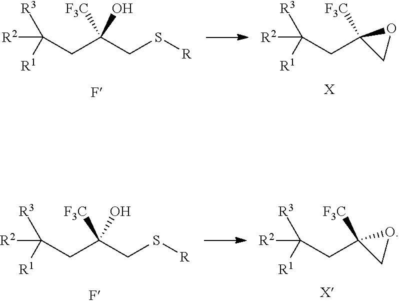

Another aspect of the invention includes a process for stereoselective synthesis of a compound of Formula (X) or Formula (X′), wherein:

R¹ is an aryl group substituted with one to three substituent groups,
wherein each substituent group of R¹ is independently C₁-C₅ alkyl, C₂-C₅ alkenyl, C₂-C₅, C₁-C₅ alkoxy, C₁-C₅ alkoxycarbonyl, aminocarbonyl, alkylaminocarbonyl, dialkylaminocarbonyl, halogen, hydroxy, carboxy, cyano, trifluoromethyl, trifluoromethoxy, or C₁-C₅ alkylthio wherein the sulfur atom is oxidized to sulfoxide or sulfone, and
   R² and R³ are each independently C₁-C₃ alkyl,
   the process as set forth above with R¹, R², and R³ as specified.

In an aspect of the invention, the suitable solvent of step (a) is diethyl ether, dipropyl ether, diisopropyl ether, dibutyl ether, tetrahydrofuran (THF), ethylene glycol dimethyl ether (DME), tert-butyl methyl ether (MTBE), or a mixture thereof, preferably diethyl ether or tetrahydrofuran.

In an aspect of the invention, the suitable solvent of step (b) is diethyl ether, dipropyl ether, diisopropyl ether, dibutyl ether, THF, DME, MTBE, toluene, or a mixture thereof, preferably diethyl ether or THF.

In an aspect of the invention, the suitable M of step (b) is Li or MgX, wherein X is Cl, Br, or I.

In an aspect of the invention, the suitable solvent of step (c) is diethyl ether, dipropyl ether, diisopropyl ether, dibutyl ether, THF, DME, MTBE, toluene, or a mixture thereof, preferably diethyl ether or THF.

In another aspect of the invention, the chiral sulfoxide anion source D or D′ is generated from the corresponding neutral sulfoxide precursor with a base selected from lithium diisopropylamide (LDA), sodium hexamethyldisilazide (NaHMDS), lithium hexamethyldisilazide (LiHMDS), potassium hexamethyldisilazide (KHMDS), sodium hydride, potassium hydride, n-butyllithium, methyllithium, ethyl magnesium bromide, and methylmagnesium bromide.

In yet another aspect of the invention, the reduction of step (d) is accomplished using a reducing agent selected from lithium aluminum hydride (LAH), diisobutyl aluminum hydride (DIBAL), or a 65 wt. % solution of sodium bis(2-methoxyethoxy)aluminum hydride in toluene (Red-Al®), or using other conditions selected from trifluoroacetic acid anhydride/sodium iodide (P. Bravo et al., J. Org. Chem., 1992, 57, 2726), trifluoroacetic acid anhydride/2,4,6-trimethylpyridine (P. Bravo et al., J. Org. Chem., 1990, 55, 4216), or hydrogen chloride in ethanol (J. L. García Ruano et al., J. Org. Chem. 1994, 59, 533).

In still another aspect of the invention, when the reduction step (d) is performed with a reducing agent, such as the aluminum hydride reagents listed above, a suitable solvent is diethyl ether, toluene, THF, MTBE, hexanes, or a mixture thereof.

In another aspect of the invention, an alkylating agent is used in step (e), preferably an alkyl halide selected from methyl iodide, methyl bromide, and ethyl iodide, or a trialkyloxonium reagent selected from trimethyloxonium tetrafluoroborate, trimethyloxonium hexachloroantimonate, triethyloxonium tetrafluoroborate, triethyloxonium hexafluorophosphate, and triethyloxonium hexachloroantimonate.

In yet another aspect of the invention, the cyclization of step (e) is accomplished with a suitable organic or inorganic base, preferably triethylamine (TEA), diisopropylethylamine (DIEA), pyridine, lutidine, sodium hydride, potassium hydride, potassium carbonate, or sodium carbonate.

In still another aspect of the invention, the suitable solvent of step (e) is dichloromethane, chloroform, dichloroethane, THF, diethyl ether, toluene, benzene, ethyl acetate, or a mixture thereof.

In yet another aspect of the invention, this process can be used to prepare the enantiomeric epoxide.

It should be noted that the invention should be understood to include none, some, or all of these various aspects in various combination.

### DETAILED DESCRIPTION OF THE INVENTION

### Definition of Terms and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

### A. Chemical Nomenclature, Terms, and Conventions

In the groups, radicals, or moieties defined below, the number of carbon atoms is often specified preceding the group, for example, C₁-C₁₀ alkyl means an alkyl group or radical having 1 to 10 carbon atoms. The term “lower” applied to any carbon-containing group means a group containing from 1 to 8 carbon atoms, as appropriate to the group (i.e., a cyclic group must have at least 3 atoms to constitute a ring). In general, for groups comprising two or more subgroups, the last named group is the radical attachment point, for example, “alkylaryl” means a monovalent radical of the formula Alk-Ar-, while “arylalkyl” means a monovalent radical of the formula Ar-Alk- (where Alk is an alkyl group and Ar is an aryl group). Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups.

The terms “alkyl” or “alkyl group” mean a branched or straight-chain saturated aliphatic hydrocarbon monovalent radical. This term is exemplified by groups such as methyl, ethyl, n-propyl, 1-methylethyl (isopropyl), n-butyl, n-pentyl, 1,1-dimethylethyl (tert-butyl), and the like. It may be abbreviated “Alk”.

The terms “alkenyl” or “alkenyl group” mean a branched or straight-chain aliphatic hydrocarbon monovalent radical containing at least one carbon-carbon double bond. This term is exemplified by groups such as ethenyl, propenyl, n-butenyl, isobutenyl, 3-methylbut-2-enyl, n-pentenyl, heptenyl, octenyl, decenyl, and the like.

The terms “alkynyl” or “alkynyl group” mean a branched or straight-chain aliphatic hydrocarbon monovalent radical containing at least one carbon-carbon triple bond. This term is exemplified by groups such as ethynyl, propynyl, n-butynyl, 2-butynyl, 3-methylbutynyl, n-pentynyl, heptynyl, octynyl, decynyl, and the like.

The terms “alkylene” or “alkylene group” mean a branched or straight-chain saturated aliphatic hydrocarbon divalent radical having the specified number of carbon atoms. This term is exemplified by groups such as methylene, ethylene, propylene, n-butylene, and the like, and may alternatively and equivalently be denoted herein as -(alkyl)-.

The terms “alkenylene” or “alkenylene group” mean a branched or straight-chain aliphatic hydrocarbon divalent radical having the specified number of carbon atoms and at least one carbon-carbon double bond. This term is exemplified by groups such as ethenylene, propenylene, n-butenylene, and the like, and may alternatively and equivalently be denoted herein as -(alkylenyl)-.

The terms “alkynylene” or “alkynylene group” mean a branched or straight-chain aliphatic hydrocarbon divalent radical containing at least one carbon-carbon triple bond. This term is exemplified by groups such as ethynylene, propynylene, n-butynylene, 2-butynylene, 3-methylbutynylene, n-pentynylene, heptynylene, octynylene, decynylene, and the like, and may alternatively and equivalently be denoted herein as -(alkynyl)-.

The terms “alkoxy” or “alkoxy group” mean a monovalent radical of the formula AlkO-, where Alk is an alkyl group. This term is exemplified by groups such as methoxy, ethoxy, propoxy, isopropoxy, butoxy, sec-butoxy, tert-butoxy, pentoxy, and the like.

The terms “alkoxycarbonyl” or “alkoxycarbonyl group” mean a monovalent radical of the formula AlkO-C(O)—, where Alk is alkyl. Exemplary alkoxycarbonyl groups include methoxycarbonyl, ethoxycarbonyl, tert-butyloxycarbonyl, and the like.

The term “alkoxycarbonylamino” or “alkoxycarbonylamino group” mean a monovalent radical of the formula ROC(O)NH—, where R is lower alkyl.

The terms “alkylcarbonylamino” or “alkylcarbonylamino group” or “alkanoylamino” or “alkanoylamino groups” mean a monovalent radical of the formula AlkC(O)NH—, where Alk is alkyl. Exemplary alkylcarbonylamino groups include acetamido (CH₃C(O)NH—).

The terms “alkylaminocarbonyloxy” or “alkylaminocarbonyloxy group” mean a monovalent radical of the formula AlkNHC(O)O—, where Alk is alkyl.

The terms “amino” or “amino group” mean an —NH₂ group.

The terms “alkylamino” or “alkylamino group” mean a monovalent radical of the formula (Alk)NH—, where Alk is alkyl. Exemplary alkylamino groups include methylamino, ethylamino, propylamino, butylamino, tert-butylamino, and the like.

The terms “dialkylamino” or “dialkylamino group” mean a monovalent radical of the formula (Alk)(Alk)N—, where each Alk is independently alkyl. Exemplary dialkylamino groups include dimethylamino, methylethylamino, diethylamino, dipropylamino, ethylpropylamino, and the like.

The terms “aminocarbonyl”, “alkylaminocarbonyl” or “dialkylaminocarbonyl” mean a monovalent radical of the formula R₂NC(O)—, where the R is independently hydrogen or alkyl.

The terms “substituted amino” or “substituted amino group” mean a monovalent radical of the formula —NR₂, where each R is independently a substituent selected from hydrogen or the specified substituents (but where both Rs cannot be hydrogen). Exemplary substituents include alkyl, alkanoyl, aryl, arylalkyl, cycloalkyl, heterocyclyl, heteroaryl, heteroarylalkyl, and the like.

The terms “alkoxycarbonylamino” or “alkoxycarbonylamino group” mean a monovalent radical of the formula AlkOC(O)NH—, where Alk is alkyl.

The terms “halogen” or “halogen group” mean a fluoro, chloro, bromo, or iodo group.

The term “halo” means one or more hydrogen atoms of the group are replaced by halogen groups.

The terms “alkylthio” or “alkylthio group” mean a monovalent radical of the formula AlkS-, where Alk is alkyl. Exemplary groups include methylthio, ethylthio, n-propylthio, isopropylthio, n-butylthio, and the like.

The terms “sulfonyl” or “sulfonyl group” mean a divalent radical of the formula —SO₂—.

The terms “aminosulfonyl”, “alkylaminosulfonyl” and “dialkylaminosulfonyl” mean a monovalent radical of the formula R₂N—SO₂—, wherein R is independently hydrogen or alkyl

The terms “aryl” or “aryl group” mean an aromatic carbocyclic monovalent or divalent radical of from 6 to 14 carbon atoms having a single ring (e.g., phenyl or phenylene) or multiple condensed rings (e.g., naphthyl or anthranyl). Unless otherwise specified, the aryl ring may be attached at any suitable carbon atom which results in a stable structure and, if substituted, may be substituted at any suitable carbon atom which results in a stable structure. Exemplary aryl groups include phenyl, naphthyl, anthryl, phenanthryl, indanyl, indenyl, biphenyl, and the like. It may be abbreviated “Ar”.

The term “compounds of the invention” and equivalent expressions are meant to embrace compounds of Formula (I) as herein described, including the tautomers, the prodrugs, the salts, particularly the pharmaceutically acceptable salts, and the solvates and hydrates thereof, where the context so permits. Certain compounds of Formula (I) are disclosed in U.S. Pat. No. 6,903,215, U.S. Patent Application Publication No. 2005/0176706, and U.S. Patent Application Publication No. 2009/0325988, and these are each incorporated by reference in their entireties. In general and preferably, the compounds of the invention and the formulas designating the compounds of the invention are understood to only include the stable compounds thereof and exclude unstable compounds, even if an unstable compound might be considered to be literally embraced by the compound formula. Similarly, reference to intermediates, whether or not they themselves are claimed, is meant to embrace their salts and solvates, where the context so permits. For the sake of clarity, particular instances when the context so permits are sometimes indicated in the text, but these instances are purely illustrative and it is not intended to exclude other instances when the context so permits.

The terms “optional” or “optionally” mean that the subsequently described event or circumstances may or may not occur, and that the description includes instances where the event or circumstance occurs and instances in which it does not. For example, “optionally substituted aryl” means that the aryl radical may or may not be substituted and that the description includes both substituted aryl radicals and aryl radicals having no substitution.

The terms “stable compound” or “stable structure” mean a compound that is sufficiently robust to survive isolation to a useful degree of purity from a reaction mixture, and formulation into an efficacious therapeutic or diagnostic agent. For example, a compound which would have a “dangling valency” or is a carbanion is not a compound contemplated by the invention.

The term “substituted” means that any one or more hydrogens on an atom of a group or moiety, whether specifically designated or not, is replaced with a selection from the indicated group of substituents, provided that the atom's normal valency is not exceeded and that the substitution results in a stable compound. If a bond to a substituent is shown to cross the bond connecting two atoms in a ring, then such substituent may be bonded to any atom on the ring. When a substituent is listed without indicating the atom via which such substituent is bonded to the rest of the compound, then such substituent may be bonded via any atom in such substituent. For example, when the substituent is piperazinyl, piperidinyl, or tetrazolyl, unless specified otherwise, such piperazinyl, piperidinyl, or tetrazolyl group may be bonded to the rest of the compound of the invention via any atom in such piperazinyl, piperidinyl, or tetrazolyl group. Generally, when any substituent or group occurs more than one time in any constituent or compound, its definition on each occurrence is independent of its definition at every other occurrence. Such combinations of substituents and/or variables, however, are permissible only if such combinations result in stable compounds.

In a specific embodiment, the term “about” or “approximately” means within 20%, preferably within 10%, and more preferably within 5% of a given value or range.

The yield of each of the reactions described herein is expressed as a percentage of the theoretical yield.

### EXPERIMENTAL EXAMPLES

The invention provides processes for making compounds of Formula (X) or (X′). In all schemes, unless specified otherwise, R¹ to R³ in the formulas below have the meanings of R¹ to R³ in the Summary of the Invention section. Intermediates used in the preparation of compounds of the invention are either commercially available or readily prepared by methods known to those skilled in the art.

The epoxide of Formula (II) is a key intermediate in the synthesis of certain racemic compounds of Formula (I), as described in Daniel Kuzmich et al., U.S. Patent Application Pub. No. 2004/0162321, which is hereby incorporated by reference. Treatment of the epoxide of Formula (II) with the nucleophile R⁵H, in the presence of base opens the epoxide to provide racemic (I) as shown below in Scheme I

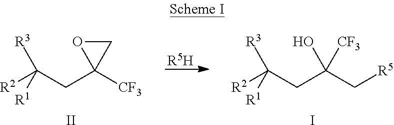

The stereoselective synthesis of a single enantiomer of epoxide (II) is carried out as shown in Scheme II below.

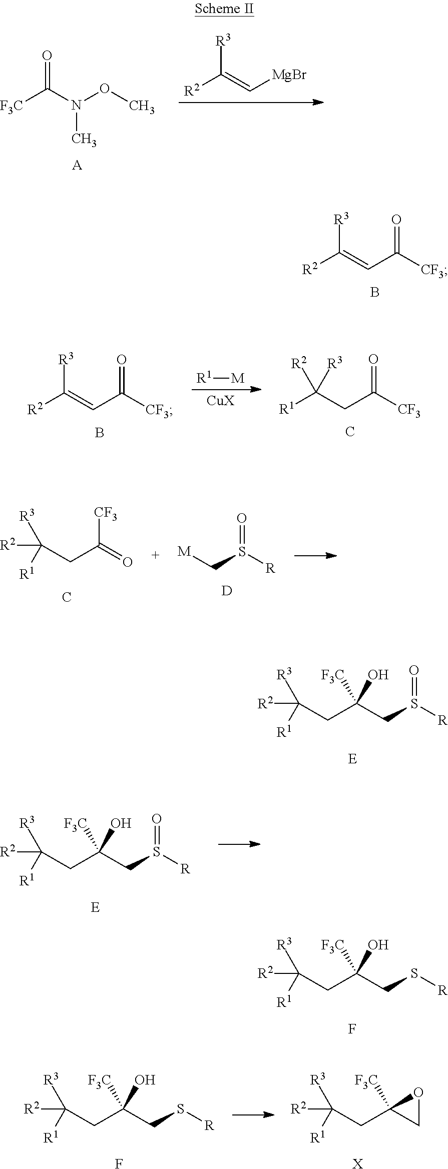

As illustrated in Scheme II, reacting the trifluoroacetamide of formula A with a vinyl magnesium bromide bearing R² and R³ in a suitable solvent to provide the trifluoromethylenone of formula B. Reacting the trifluoromethylenone of formula B with a suitable organocopper reagent generated from an organometallic reagent R⁵R⁴M where M is Li or MgX and a copper salt CuX, where X is Cl, Br, I, or CN, in a suitable solvent to form the ketone of formula C.

As illustrated in Scheme II, reacting the ketone of formula C with a chiral sulfoxide anion source D in the presence of a suitable base, such as LDA, in a suitable solvent, such as THF, provides a compound of formula E. Reduction of the sulfoxide of formula E with suitable reducing agents affords the compound of formula F. Reaction of the compound of formula F with reagents such as trimethyloxonium tetrafluoroborate in a suitable solvent, such as dichloromethane, in the presence of a suitable base, such as potassium carbonate, provides epoxide of Formula (X) which is a single enantiomer of epoxide (II). The analogous reaction can be performed to make the other enantiomer of Formula (X′).

Preparation of the Desired Enantiomer of Formula (I) can then be Achieved by Reaction of the compound of Formula (X) or Formula (X′), which is enantiomerically pure epoxide of formula (II), with the appropriate nitrogen, oxygen, sulfur, or carbon nucleophile (R⁵H).

Optimum reaction conditions and reaction times may vary depending on the particular reactants used. Unless otherwise specified, solvents, temperatures, pressures, and other reaction conditions may be readily selected by one of ordinary skill in the art. Furthermore, if the substituent groups on R¹ to R³ are incompatible under the reaction conditions of the process, protection/deprotection of these groups may be carried out, as required, using reagents and conditions readily selected by one of ordinary skill in the art, see, for example, T. W. Greene and P. G. M. Wuts, Protective Groups in Organic Synthesis, New York: John Wiley & Sons (1999) and references cited therein. For example, a hydroxyl group can be protected as methyl ether and be deprotected at an appropriate stage with reagents, such as boron tribromide in dichloromethane. Specific procedures are provided in the Experimental Examples section. Typically, reaction progress may be monitored by high performance liquid chromatography (HPLC) or thin layer chromatography (TLC), if desired, and intermediates and products may be purified by chromatography on silica gel by recrystallization and/or distillation.

### Synthetic Examples

The following are representative examples that illustrate the process of the invention. HPLC used to determine diastereoselectivity were done on a Supelco SUPELCOSIL™ ABZ+Plus column (4.6 mm×10 cm) eluting with a gradient of 5% acetonitrile/95% water/0.05% TFA to 100% acetonitrile/0.05% TFA over 15 minutes and then held at 100% acetonitrile/0.05% TFA for 5 minutes. References to concentration or evaporation of solutions refer to concentration on a rotary evaporator.

### Example 1

### Synthesis of 2-[1,1-Dimethyl-2-((R)-2-trifluoromethyloxiranyl)ethyl]-benzoic acid

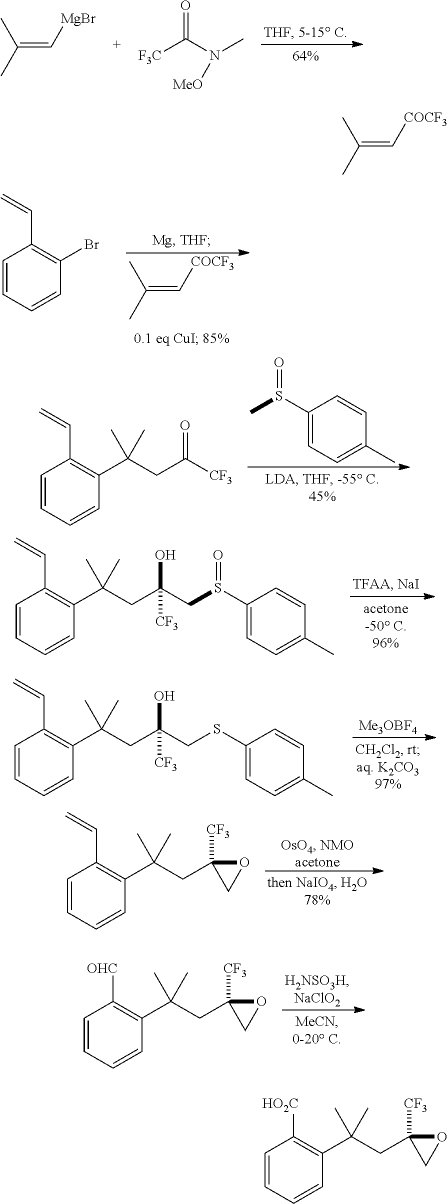

### 1,1,1-Trifluoro-4-methylpent-3-en-2-one

2-Methyl-1-propenylmagnesium bromide (0.5M in THF, 2.4 L, 1.2 mol) was cooled to 5° C. N-methoxy-N-methyl trifluoroacetamide (130.8 mL, 1.08 mol) was added dropwise over 30 minutes to the Grignard solution. The reaction mixture was allowed to stir at 20° C.-25° C. for 3 hours. The reaction mixture was cooled to 5° C. and treated dropwise over 30 minutes with 300 mL of concentrated HCl, keeping the temperature between 5° C.-20° C. The reaction was further diluted with 900 mL of water and 600 mL of dodecane, and the layers were separated. The organic phase was washed four times with a solution of 975 mL of water and 225 mL of methanol (MeOH), then with 1 L water, and finally dried over 4A molecular sieves (50 g) for 12 hours. The solution was filtered away from the molecular sieves and distilled at 150 mmHg (bath temperature up to 135° C.) to give 1,1,1-trifluoro-4-methylpent-3-en-2-one (245.9 g assayed, 64% yield) as a yellow solution in THF (˜60-70 wt. %).

### 1,1,1-Trifluoro-4-methyl-4-(2-vinylphenyl)pentan-2-one

To magnesium metal turnings (14.0 g, 0.565 mol) in 300 mL of THF was added iodine (150 mg). The mixture was stirred for 30 minutes at room temperature. Approximately 30 mL of a solution of 2-bromostyrene (101.7 g, 0.539 mol) in 100 mL of THF was added. The temperature of the reaction mixture rose within 5 minutes to 57° C. with concurrent disappearance of the iodine color. The remainder of the 2-bromostyrene solution was added dropwise over 1 hour and 40 minutes, keeping the temperature of the reaction mixture between 59° C.-67° C. The reaction mixture was heated an additional 1 hour at 67° C.-69° C., cooled to room temperature, and titrated as 0.965 M.

In a separate flask, copper iodide (CuI) (1.0 g, 5.20 mmol), THF (200 mL), and 1,1,1-trifluoro-4-methylpent-3-en-2-one (90.45 g, 74.3 wt. %, 0.442 mol) were added. The yellow cloudy slurry was cooled to −24° C. and treated over 1 hour with the Grignard solution, keeping the temperature below −10° C. during the addition. The reaction mixture was stirred at −10° C. to −5° C. for an additional 2 hours, quenched with 300 mL of saturated ammonium chloride (NH₄Cl) solution followed by 200 mL of ethyl acetate, stirred for 10 minutes, and the layers were separated. The organic phase was washed with 200 mL of saturated NH₄Cl solution, then with 200 mL of brine, and finally concentrated in vacuo to a yellow liquid. This crude product was taken up in 250 mL of heptane, filtered through a pad of CELITE® filter aid, and concentrated in vacuo to give 1,1,1-trifluoro-4-methyl-4-(2-vinylphenyl)-pentan-2-one (125.7 g, 76.3 wt. %, 85% yield) as a light yellow liquid.

### (S)-1,1,1-Trifluoro-4-methyl-2-(toluene-4-(R)-sulfinylmethyl)-4-(2-vinylphenyl)pentan-2-ol

A solution of 1-((R)-methanesulfinyl)-4-methylbenzene (100.5 g, 0.652 mol) in 700 mL of THF was cooled to −65° C. Lithium diisopropylamide (LDA) (1.54 M in cyclohexane, 457.0 mL, 0.704 mol) was added dropwise over 1 hour and 50 minutes. The reaction mixture was stirred for 0.5 hour at −65° C. A solution of 1,1,1-trifluoro-4-methyl-4-(2-vinylphenyl)pentan-2-one (206.55 g, 78.5 wt. %, 0.632 mol) in 400 mL of THF was added dropwise over 2 hour and 45 minutes, keeping the temperature below −65° C. The reaction mixture was stirred for 30 minutes at −65° C., quenched with 400 mL of half-saturated brine, and allowed to warm to room temperature. The layers were separated and the aqueous phase extracted with 350 mL of EtOAc. The combined organic phases were concentrated in vacuo to a thick yellow oil. Chromatography on silica gel (5-50% EtOAc in hexanes) gave the desired sulfoxide (117.0 g, 45%) as a white solid.

### (S)-1,1,1-Trifluoro-4-methyl-2-(toluene-4-thiomethyl)-4-(2-vinylphenyl)pentan-2-ol

A sodium carbonate solution was prepared by dissolving sodium carbonate (90.4 g) in 270 mL of water. A sodium sulfite solution was prepared by dissolving sodium sulfite (53.8 g) in 220 mL of water. A mixture of the above sulfoxide (70.0 g, 171 mmol) and sodium iodide (76.7 g, 512 mmol) in 910 mL of acetone was agitated for 30 minutes at Tᵢₙₜ=20° C.-22° C. to afford a homogeneous solution. The solution was slowly cooled over 0.5 hours to Tᵢₙₜ=−57° C. to afford a mixture that could be stirred. To this mixture was added trifluoroacetic anhydride (119 mL, 179 g, 853 mmol) dropwise at a rate to maintain Tᵢₙₜ=−57° C. to −50° C. over 25 minutes. The resulting dark green-blue mixture was agitated at Tᵢₙₜ=−60° C. to −52° C. for 20 minutes, at which point HPLC (220 nm) showed complete consumption of starting sulfoxide. The reaction was quenched by the addition of the sodium sulfite solution at a rate to maintain Tᵢₙₜ<−20° C. and required 10 minutes. The reaction was warmed to Tᵢₙₜ=−15° C. The mixture was charged with the sodium carbonate solution at a rate to maintain Tᵢₙₜ=−5° C. to 5° C. and to control the gas evolution. The sodium carbonate addition required 40 minutes. The mixture was diluted with 420 mL of hexanes and allowed to warm to Tᵢₙₜ=20° C. and stirred at this temperature for 20 minutes. The aqueous layer was separated and extracted with two 420 mL portions of hexanes. The combined organic portions were dried over magnesium sulfate (MgSO₄), filtered, and concentrated in vacuo to the desired sulfide as a thick oil in 94 wt. % (68.6 g, 96%).

### (R)-2-[2-Methyl-2-(2-vinylphenyl)propyl]-2-trifluoromethyloxirane

A potassium carbonate solution was prepared by dissolving potassium carbonate (68 g) in 210 mL of water. The sulfide obtained form previous step (94 wt. %, 68.1 g, 162 mmol) was dissolved in 100 mL of methylene chloride (CH₂Cl₂) and was added over 3 minutes to a suspension of trimethyloxonium tetrafluoroborate (29.4 g, 195 mmol) in 400 mL of methylene chloride at Tᵢₙₜ=20° C.-22° C. under argon. The resulting orange heterogeneous reaction was stirred at the above temperature for 3 hours, at which point HPLC (220 nm) showed complete consumption of starting sulfide. The reaction was charged with the potassium carbonate solution over 1 minutes. The mixture was agitated at Tᵢₙₜ=20° C.-22° C. for 1 hour, at which point HPLC (220 nm) showed complete consumption of the intermediate. The reaction was diluted with 400 mL of water and extracted with one 400 mL and two 200 mL portions of chloroform. The combined organic extracts were dried with magnesium sulfate, filtered, and concentrated in vacuo to an oil. The oil was taken up into 250 mL of methanol and treated with hydrogen peroxide (30 wt. % in water, 83 mL, 811 mmol) dropwise at a rate to maintain Tᵢₙₜ=20° C.-25° C. The reaction was placed into a water bath and stirred at Tᵢₙₜ=20° C.-27° C. for 7 hours, at which point complete oxidation of the by product methyl p-tolyl sulfide to methyl p-tolyl sulfoxide was observed by HPLC (220 nm). The reaction was diluted with 450 mL of hexanes and washed with two 400 mL and one 200 mL portions of water. The aqueous washes were discarded and not combined with the subsequent washes. The organic portion was washed with two 200 mL portions of an aqueous sodium sulfite solution (10 wt. %), dried over magnesium sulfate, filtered, and concentrated in vacuo to the desired epoxide (R)-2-[2-methyl-2-(2-vinylphenyl)propyl]-2-trifluoromethyloxirane as an oil in 95 wt. % (44.8 g, 97%).

### 2-[1,1-Dimethyl-2-((R)-2-trifluoromethyloxiranyl)ethyl]benzaldehyde

To a solution of (R)-2-[2-methyl-2-(2-vinylphenyl)propyl]-2-trifluoromethyloxirane (94.7 wt. %, 44.1 g, 155 mmol) in 415 mL of acetone was added N-methylmorpholine-N-oxide (36.2 g, 309 mmol) followed by osmium tetraoxide (2.5 wt. % in tert-butanol, 38.8 mL, 31.44 g, 3.09 mmol) at Tᵢₙₜ=20° C.-22° C. under nitrogen. The resulting dark orange solution was agitated for 2.5 hours, at which point complete consumption of the starting epoxide was observed by HPLC (220 nm). Sodium periodate (49.6 g, 232 mmol) followed by 166 mL of water were added to the reaction at Tᵢₙₜ=20° C.-22° C. The mixture was agitated for 1.8 hours, at which point additional sodium periodate (8.3 g, 39 mmol) was added to the reaction. The mixture was agitated for 30 minutes, at which point complete consumption of the intermediate diol was observed by HPLC (220 nm). The reaction was diluted with 460 mL of ethyl acetate and washed with 460 mL of water, 51 mL of brine (5 wt. %). The aqueous portions were combined and extracted with 370 mL of ethyl acetate. The combined organic portions were washed with 515 mL of brine (5 wt. %), two 414 mL portions of aqueous sodium sulfite (10 wt. %), dried over magnesium sulfate, filtered, and concentrated in vacuo to the desired aldehyde 2-[1,1-dimethyl-2-((R)-2-trifluoromethyloxiranyl)ethyl]benzaldehyde as an oil in 72 wt % (46 g, 78%).

### 2-[1,1-Dimethyl-2-((R)-2-trifluoromethyloxiranyl)ethyl]benzoic acid

Sulfamic acid (17.5 g, 176 mmol) dissolved in 120 mL of water was added to a solution of 2-[1,1-dimethyl-2-((R)-2-trifluoromethyloxiranyl)ethyl]benzaldehyde (72 wt. %, 44.4 g, 117 mmol) in 200 mL of acetonitrile at Tᵢₙₜ=0° C.-2° C. Sodium chlorite (19.9 g, 176 mmol) dissolved in 120 mL of water was added to the above solution dropwise over 10 minutes at a rate to maintain Tᵢₙₜ=10° C.-19° C. The resulting yellow mixture was agitated for 5 minutes, at which point the reaction was warmed to Tᵢₙₜ=17° C. in 30 minutes. HPLC (220 nm) showed >20:1 by area of the desired carboxylic acid 2-[1,1-dimethyl-2-((R)-2-trifluoromethyloxiranyl)ethyl]benzoic acid to starting aldehyde. The reaction was diluted with 350 mL of ethyl acetate, washed with two 100 mL portions of brine (10 wt. %), dried over magnesium sulfate, filtered, and concentrated in vacuo to the desired carboxylic acid as an oil.

2-[1,1-Dimethyl-2-((R)-2-trifluoromethyloxiranyl)ethyl]-5-fluorobenzoic acid was synthesized in an analogous manner.

What is claimed is:

1. A process for stereoselective synthesis of a compound of Formula (X) or Formula (X′) 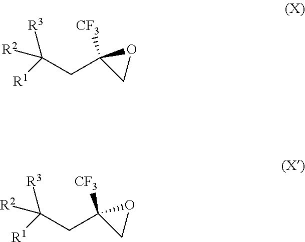
wherein:
R¹ is an aryl group substituted with one to three substituent groups,
wherein each substituent group of R¹ is independently C₁-C₅ alkyl, C₂-C₅ alkenyl, C₂-C₅ alkynyl, C₁-C₅ alkoxy, C₁-C₅ alkoxycarbonyl, aminocarbonyl, alkylaminocarbonyl, dialkylaminocarbonyl, C₁-C₅ alkoxycarbonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, or C₁-C₅ alkylthio wherein the sulfur atom is oxidized to sulfoxide or sulfone, and
R² and R³ are each independently hydrogen or C₁-C₅ alkyl,
the process comprising:
a) reacting the trifluoroacetamide of Formula (A) wherein R′ and R″ are each independently C₁-C₅ alkyl optionally substituted with O or N (e.g., morpholine amide or Weinreb amide) with a vinyl magnesium bromide bearing R² and R³ in a suitable solvent to provide the trifluoromethylenone of Formula (B) 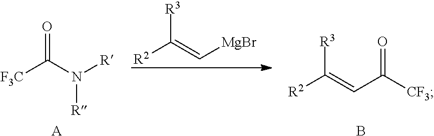
(b) reacting the trifluoromethylenone of Formula (B) with a suitable organocopper reagent generated from an organometallic reagent R⁵R⁴M where M is Li or MgX and a copper salt CuX, where X is Cl, Br, I, or CN, in a suitable solvent to form the ketone of Formula (C) 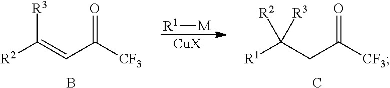
(c) reacting the ketone of Formula (C) with a chiral sulfoxide anion source (D) or (D′), where R is an alkyl or aryl group and M is a counter-cation, in a suitable solvent to prepare a compound of Formula (E) or (E′), respectively 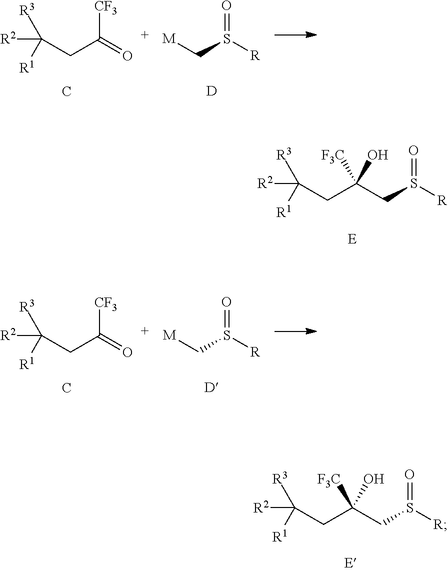
(d) reducing the sulfoxide of Formula (E) or (E′) in a suitable solvent to obtain the compound of Formula (F) or (F′), respectively 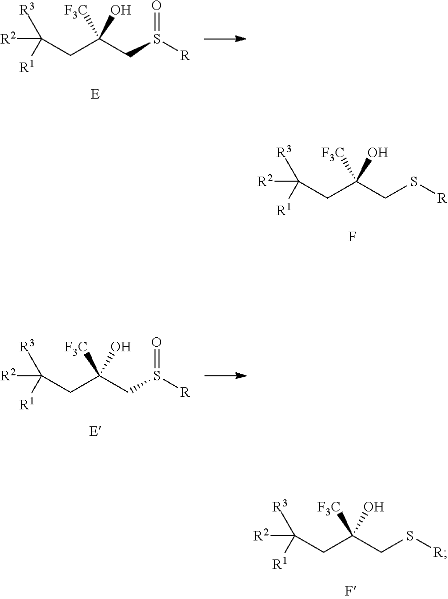
and
(e) cyclizing the compound of Formula (F) or (F′) in a suitable solvent to form the epoxide compound of Formula (X) or Formula (X′), respectively

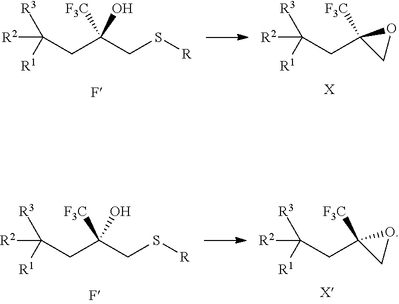

2. The process according to claim 1, wherein:
R¹ is an aryl group substituted with one to three substituent groups,
wherein each substituent group of R¹ is independently C₁-C₅ alkyl, C₂-C₅ alkenyl, C₂-C₅, C₁-C₅ alkoxy, C₁-C₅ alkoxycarbonyl, aminocarbonyl, alkylaminocarbonyl, dialkylaminocarbonyl, halogen, hydroxy, carboxy, cyano, trifluoromethyl, trifluoromethoxy, or C₁-C₅ alkylthio wherein the sulfur atom is oxidized to sulfoxide or sulfone, and
R² and R³ are each independently C₁-C₃ alkyl.

3. The process according to claim 1, wherein the suitable solvent of step (a) is diethyl ether, dipropyl ether, diisopropyl ether, dibutyl ether, THF, DME, MTBE, or a mixture thereof.

4. The process according to claim 3, wherein the suitable solvent of step (a) is diethyl ether or THF.

5. The process according to claim 1, wherein the suitable solvent of step (b) is diethyl ether, dipropyl ether, diisopropyl ether, dibutyl ether, THF, DME, MTBE, toluene, or a mixture thereof.

6. The process according to claim 5, wherein the suitable solvent of step (b) is diethyl ether or THF.

7. The process according to claim 1, wherein the suitable M of step (b) is Li or MgX, wherein X is Cl, Br, or I.

8. The process according to claim 1, wherein the suitable solvent of step (c) is diethyl ether, dipropyl ether, diisopropyl ether, dibutyl ether, THF, DME, MTBE, toluene, or a mixture thereof.

9. The process according to claim 8, wherein the suitable solvent of step (c) is diethyl ether or THF.

10. The process according to claim 1, wherein the chiral sulfoxide anion source D or D′ is generated from the corresponding neutral sulfoxide precursor with a base selected from LDA, NaHMDS, LiHMDS, KHMDS, sodium hydride, potassium hydride, n-butyllithium, methyllithium, ethyl magnesium bromide, and methylmagnesium bromide.

11. The process according to claim 1, wherein the reduction of step (d) is accomplished using a reducing agent selected from LAH, DIBAL, or a 65 wt. % solution of sodium bis(2-methoxyethoxy)aluminum hydride in toluene (Red-Al®), or using other conditions selected from trifluoroacetic acid anhydride/sodium iodide, trifluoroacetic acid anhydride/2,4,6-trimethylpyridine, or hydrogen chloride in ethanol.

12. The process according to claim 11, wherein the suitable solvent is diethyl ether, toluene, THF, MTBE, hexanes, or a mixture thereof.

13. The process according to claim 1, wherein an alkylating agent is used in step (e).

14. The process according to claim 13, wherein the alkylating agent is an alkyl halide or trialkyloxonium reagent.

15. The process according to claim 14, wherein the alkylating agent is methyl iodide, methyl bromide, ethyl iodide, trimethyloxonium tetrafluoroborate, trimethyloxonium hexachloroantimonate, triethyloxonium tetrafluoroborate, triethyloxonium hexafluorophosphate, or triethyloxonium hexachloroantimonate.

16. The process according to claim 1, wherein the cyclization of step (e) is accomplished with a suitable organic or inorganic base.

17. The process according to claim 16, wherein the cyclization of step (e) is accomplished with TEA, DIEA, pyridine, lutidine, sodium hydride, potassium hydride, potassium carbonate, or sodium carbonate.

18. The process according to claim 1, wherein the suitable solvent of step (e) is dichloromethane, chloroform, dichloroethane, THF, diethyl ether, toluene, benzene, ethyl acetate, or a mixture thereof.
